(19) United States
(12) Patent Application Publication
(10) Pub. No.: US 2011/0028703 A1
(43) Pub. Date: Feb. 3, 2011

(54) METHOD AND APPARATUS FOR SUSPENDING MAGNETIC MICROPARTICLES

(75) Inventors: Jennifer A. Hollenstein, Grayslake, IL (US); Timothy Patno, Barrington, IL (US); Tom Westberg, Gurnee, IL (US); Melissa Chambers, Darien, IL (US); Steve Maltas, Palatine, IL (US)

Correspondence Address: GREGORY T. PLETTA;Nanosphere, Inc., 4088 Commerical Avenue, Northbrook, IL, 60062-1829, US

(73) Assignee: Nanosphere, Inc., Northbrook, IL (US)

(21) Appl. No.: 12/719,786

(22) Filed: Mar. 8, 2010

Related U.S. Application Data
(60) Provisional application No. 61/158,300, filed on Mar. 6, 2009.

(51) Int. Cl. C07H 1/06

(52) U.S. Cl. 536/23.1

(57) ABSTRACT

A method includes applying ultrasound to a container having a plurality of magnetic particles contacted with a fluid sample having a biological material capable of binding to the magnetic particles, in an amount effective to suspend the magnetic particles in the fluid.

### CROSS-REFERENCE TO RELATED APPLICATIONS

This application claims the benefit under 35 U.S.C. 119 (e) of U.S. Provisional Application No. 61/158,300, filed on Mar. 6, 2009, which is hereby incorporated by reference in its entirety.

### TECHNICAL FIELD

The invention relates to magnetic microparticles (MMPs) and more particularly with methods of controlling MMPs in the processing of biologic material (e.g., DNA, RNA, etc.).

### BACKGROUND

MMPs are small magnetic or ferromagnetic particles (having a diameter in the area of about 1 μm) covered with a glass coating. The glass coating causes the MMPs to be relatively inert to reagents in processes used for detecting the presence of particular biologic materials. MMPs can be used to extract nucleic acids from whole blood and swab solutions. Current protocols use pipette mixing to add MMPs to samples and mix. Cells are lysed and DNA is captured on the MMPs. Following DNA capture, the MMPs are washed to remove cellular debris, proteins, residual reagents, and other contaminants. Current instruments and manual protocols require vigorous mixing with pipettes or by mechanical shaking to resuspend the MMPs to facilitate DNA capture and effective washing. Poor MMP resuspension can lead to inefficient DNA extraction and decreased DNA purity due to residual proteins, salts and other contaminants.

### SUMMARY

In one aspect, a method includes applying ultrasound to a container having a plurality of magnetic particles contacted with a fluid sample having a biological material capable of binding to the magnetic particles, in an amount effective to suspend the magnetic particles in the fluid.

### BRIEF DESCRIPTION OF THE DRAWINGS

FIG. 1 shows a perspective view of a device utilized for suspending magnetic particles, in accordance with one embodiment.

FIG. 2A is an exploded view of the device of FIG. 1.

FIG. 2B is a top perspective view of the device of FIG. 1.

FIG. 2C is a bottom perspective view of the device of FIG. 1.

FIG. 2D is a perspective view of a portion of the device of FIG. 1.

FIG. 3 is a method of suspending magnetic particles, in accordance with one embodiment.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

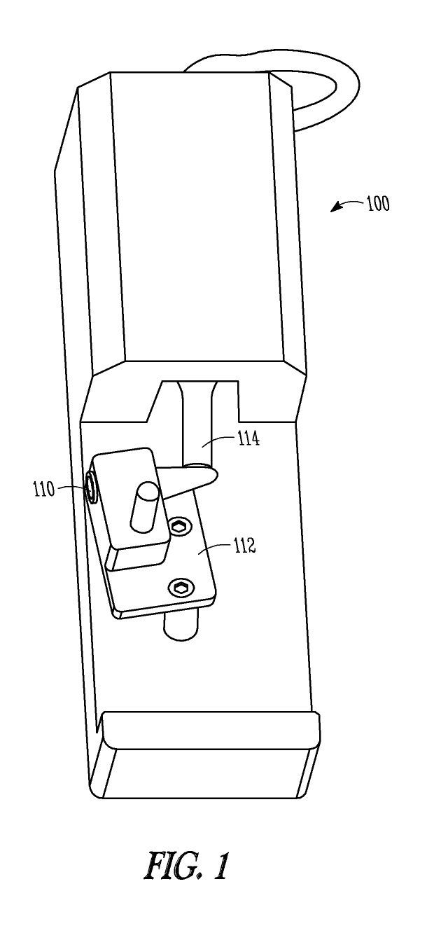

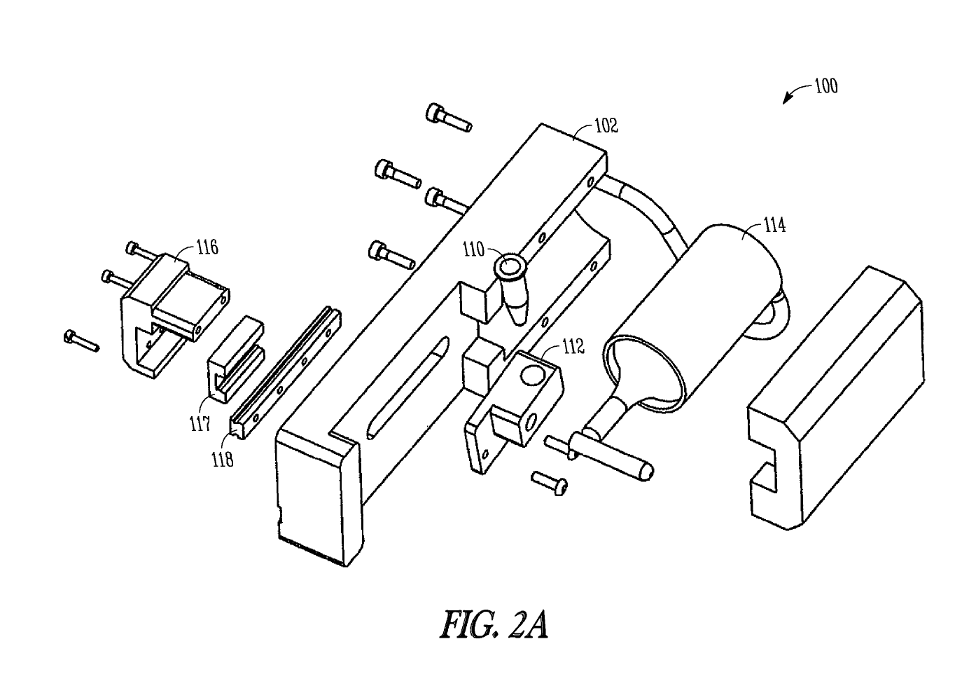

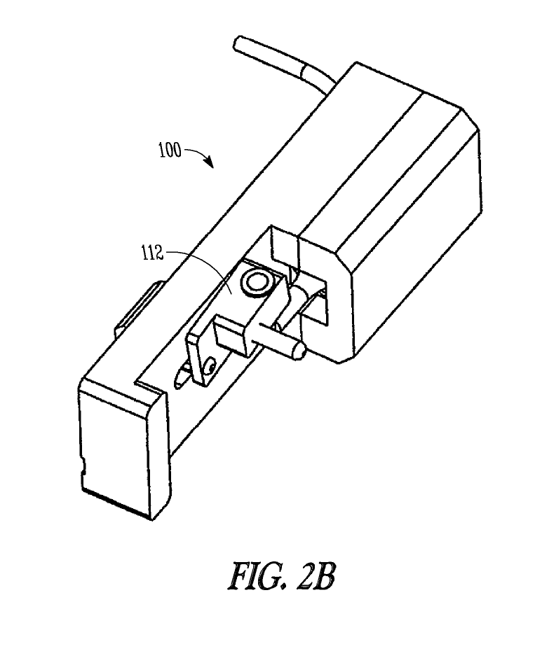

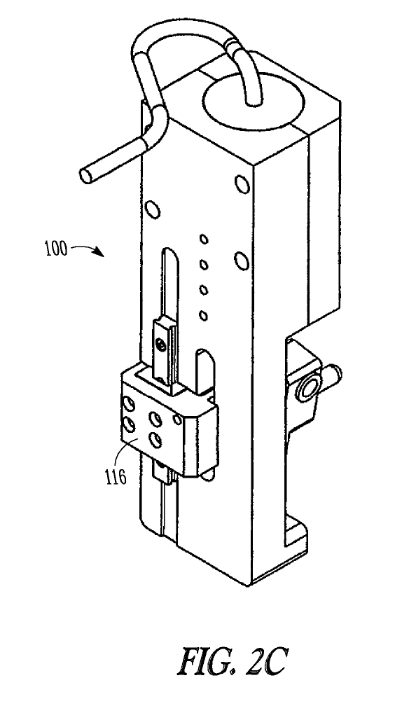

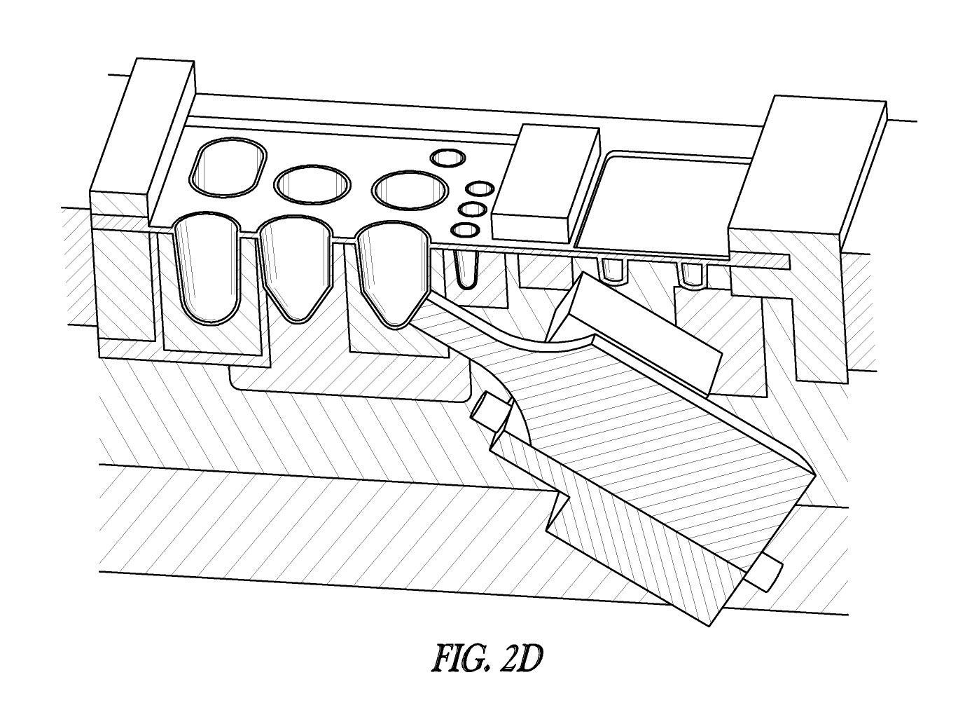

FIGS. 1-2D show a device 100 utilized for suspending magnetic particles, in accordance with one embodiment. FIG. 1 shows a perspective view of device 100. FIG. 2A is an exploded view of device 100, FIG. 2B is a top perspective view of device 100, FIG. 2C is a bottom perspective view of device 100, and FIG. 2D is another perspective view of a portion of device 100.

Device 100 generally includes a base 102 with an ultrasonic actuator 114 mounted to the base. A tube 110 for holding a sample to be processed is removably mountable to a tube holder 112. The tube holder 112 is mountable to base 102 utilizing a carrier 116, a block bearing 117, and a rail bearing 118.

In one embodiment, MMPs are placed in a container, such as tube 110, and a reagent containing a biologic material (e.g., a DNA) is added to the container. Because of the surface characteristics of the MMPs, the DNA attached itself to the surface of the MMPs. The attachment of the DNA to the MMPs may be enhanced by denaturing the sample (e.g., by heating) or by the surface preparation of the MMPs. In one embodiment ultrasonic energy delivered via ultrasonic actuator 114 at about 40 kHz is used to mix the MMPs to facilitate DNA capture.

Once a significant portion of the DNA is attached to the MMPs, the reagent and any waste material (e.g., cell membranes, contaminants, etc.) may be removed from the container. In order to remove the waste material, the MMPs and attached DNA are immobilized within the container while the container is washed.

The MMPs may be immobilized within the container by applying a magnetic field to the container. The magnetic field may be applied by placing a magnet against an outside wall of the container. Placing the magnet against the outside wall of the container causes the MMPs (and attached DNA) to coalesce against the inside wall of the container. The magnetic field is applied to the container in an amount and at a distance that concentrates or aggregates the magnetic particles in the container. Once the MMPs have been immobilized within the container, the MMPs can be washed. Washing can be accomplished by passing a wash material through the container. Washing may occur by repeatedly filling and emptying the container or by continuously passing a wash liquid through the container.

Washing may be interrupted by one or more re-suspension processes. Re-suspension may be within a wash liquid or another reagent. Re-suspension is difficult in this context (even after removal of the magnetic field) because the MMP are mutually attracted to each other and to the wall of the container.

Re-suspension in this case is accomplished by first interrupting or removing the magnet. Once the magnetic field has been interrupted, ultrasonic energy may be introduced into the container.

In one embodiment, an ultrasound power supply and transducer operating at approximately 40 kHz may be used. Some embodiments utilize a transducer operating at greater than approximately 20 kHz. Once the magnet is removed, a horn of the transducer may be placed against the outside wall of the container and the transducer is activated.

Application of the ultrasound through the wall of the container tends to break the weak bonds between the MMPs, both between each other and between the MMPs and the wall of the container. Once the bonds have been broken, the MMPs diffuse outwards from the walls into the liquid.

Once the MMPs have been re-suspended, the suspension may be allowed to equalize for some period of time in the wash liquid. Once the suspension has equalized, the magnetic field may again be applied to the container to again fix the MMPs against the walls of the container and the wash process may be repeated.

Once the MMPs have been washed, the MMPs may re-suspended in a second reagent that releases the DNA from the MMPs. Once the DNA has been released from the MMPs, the magnetic field may again be re-applied to the container to fix the MMPs to the walls of the container leaving an elute of the second reagent and DNA. The elute may be removed from the container for further processing.

In one embodiment, software can control the activation and deactivation of the 40 kHz ultrasonic horn. For example, the horn can contact the side of a high-density polyethylene well. The horn is energized in intervals to resuspend the MMPs during wash and elution steps. The amplitude and activation duration can be modified to ensure adequate resuspension.

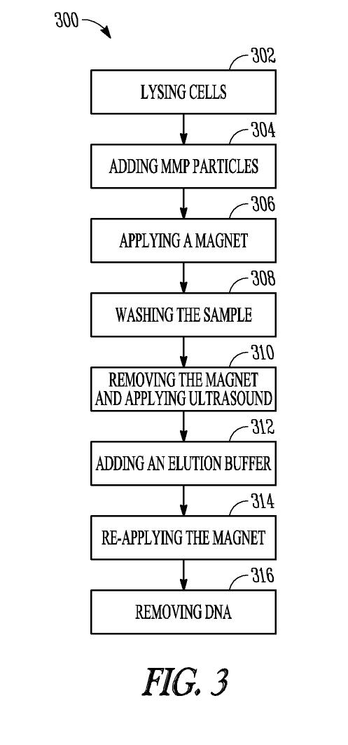

FIG. 3 is a method of suspending magnetic particles, in accordance with one embodiment.

Method 300 includes lysing cells of a sample (302); adding MMP particles to the sample (304); applying a magnet to the sample to draw the MMP cells (306); washing the sample (308); removing magnet and applying ultrasound to the sample to re-suspend the MMP particles (310); adding an elution buffer (312); re-applying the magnet (314); removing DNA (316).

Lysing the cells can include using a lysis buffer, including guanidine thiocyanate. Lysing the cells can be performed using a commercially available extractions kit based, for example, on the glass-nucleic acid binding-technique (for example MagNAPure® sold by Roche Diagnostics). The purpose of the MMP is to bind nucleic acids in the presence of chaotropic salts. The MMPs have a paramagnetic iron core encapsulated in a high surface area of silica. In some examples, the MMP particles can be any magnetic MMP particle, such as iron MMPs. The washing and magnet and ultrasound resuspension steps can be performed as discussed above. In some examples, the ultrasound can be performed using a device and method as described in U.S. Pat. No. 7,625,746, assigned to Nanosphere, Inc., and which is incorporated by reference in its entirety. The wash process (308) and ultrasound resuspension (310) can be repeated as many times as need to get a satisfactory result.

A test performed using the method described had the following results. The test resulted in a sample of 14.83 ng/μl. The purity of the 260/280 ratio (DNA/proteins) was 2.09 and the 260/230 ratio (DNA/ ration of impurities) was 0.8.

Other tests have resulted in concentrations of about 180 ng/μL and 260/280 and 260/230 of over 1.7.

In some embodiments of the methods discussed above, the ultrasound can be approximately 30 to 50 kHz. In some embodiments, the ultrasound can be approximately 35 to 45 kHz. In some embodiments, the MMP particles have a diameter of about 0.05 to about 50 microns.

The method can be utilized on fluid samples including nucleic acid, such as RNA or DNA. The fluid sample can contain lysed cells, such as eukaryotic cells.

In some examples discussed above, the ultrasonic energy may also assist with the ability to lyse cells and potentially cleave proteins.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. A method comprising;
applying ultrasound to a container having a plurality of magnetic particles contacted with a fluid sample having a biological material capable of binding to the magnetic particles, in an amount effective to suspend the magnetic particles in the fluid.

2. The method of claim 1, wherein the ultrasound is approximately 30 to 50 kHz.

3. The method of claim 2, wherein the ultrasound is approximately 35 to 45 kHz.

4. The method of claim 1, wherein the magnetic particles comprise iron.

5. The method of claim 1, wherein the magnetic particles are microparticles.

6. The method of claim 5, wherein the particles have a diameter of about 0.05 to about 50 microns.

7. The method of claim 1, wherein the fluid sample comprises nucleic acid.

8. The method of claim 7, wherein the nucleic acid comprises RNA or DNA.

9. The method of claim 1, further including prior to applying the ultrasound, applying the magnetic field to the container in an amount and at a distance that concentrates the magnetic particles in the container and washing the fluid sample in the container.

10. The method of claim 9, comprising repeating applying the magnetic field and washing the fluid sample.

11. The method of claim 1, wherein the ultrasound is applied to the container in the absence of a magnetic field capable of attracting the magnetic particles.

12. A method of processing a plurality of magnetic microparticles within a container comprising:
providing a container having a plurality of magnetic microparticles and a fluid sample suspected of having a moiety that binds to the microparticles;
applying a magnetic field to the container so as to concentrate the plurality of magnetic microparticles in a portion of the container;
removing the fluid from the container
washing the container with a fluid while applying the magnetic field; and
applying ultrasound to the container under conditions that resuspend the magnetic microparticles in the container.

13. The method of claim 12, wherein the sample is an aqueous fluid sample.

14. The method of claim 12, wherein the ultrasound is approximately 30 to 50 kHz.

15. The method of claim 14, wherein the ultrasound is approximately 35 to 45 kHz.

16. The method of claim 12, wherein the magnetic particles comprise Fe.

17. The method of claim 12, wherein the particles have a diameter of about 0.05 to about 50 microns.

18. The method of claim 12, wherein the fluid sample comprises nucleic acid.

19. The method of claim 12, wherein the fluid sample comprises lysed cells.

20. The method of claim 19, wherein the cells are eukaryotic cells.
